(19) United States
(12) Patent Application Publication
(10) Pub. No.: US 2013/0096346 A1
(43) Pub. Date: Apr. 18, 2013

(54) RESOLUTION METHODS FOR ISOLATING DESIRED ENANTIOMERS OF TAPENTADOL INTERMEDIATES AND USE THEREOF FOR THE PREPARATION OF TAPENTADOL

(71) Applicants: Mayur Devjibhai Khunt, Gujarat (IN); Sandipan Prabhurao Bondge, Maharashtra (IN); Nitin Sharadchandra Pradhan, Maharashtra (IN)

(72) Inventors: Mayur Devjibhai Khunt, Gujarat (IN); Sandipan Prabhurao Bondge, Maharashtra (IN); Nitin Sharadchandra Pradhan, Maharashtra (IN)

(73) Assignee: ACTAVIS GROUP PTC EHF, Hafnarfjordur (IS)

(21) Appl. No.: 13/580,398

(22) PCT Filed: Mar. 1, 2011
(86) PCT No.: PCT/IB11/00526

§ 371 Date: Jan. 7, 2013

(30) Foreign Application Priority Data
Mar. 5, 2010 (IN) 577/CHE/2010

(51) Int. Cl. C07C 213/10; C07C 213/00

(52) U.S. Cl. CPC C07C 213/10; C07C 213/00
USPC 564/304; 564/374

(57) ABSTRACT

Provided herein is an improved and industrially advantageous optical resolution method for resolving (2R,3R)/(2S,3S)-1-dimethylamino-3-(3-methoxyphenyl)-2-methylpentan-3-ol, and use thereof for the preparation of tapentadol or a pharmaceutically acceptable salt thereof. Provided further herein is an improved and industrially advantageous optical resolution method for resolving (2R,3R)/(2S,3S)-[3-(3-methoxyphenyl)-2-methylpentyl]-dimethylamine, and use thereof for the preparation of tapentadol or a pharmaceutically acceptable salt thereof. Disclosed also herein is an improved, commercially viable and industrially advantageous process for the preparation of tapentadol or a pharmaceutically acceptable salt thereof in high yield and purity.

### CROSS REFERENCE TO RELATED APPLICATION

This application claims the benefit of priority to Indian provisional application No. 577/CHE/2010, filed on Mar. 5, 2010, which is incorporated herein by reference in its entirety.

### FIELD OF THE DISCLOSURE

Disclosed herein is an improved and industrially advantageous optical resolution method for resolving (2R,3R)/(2S,3S)-1-dimethylamino-3-(3-methoxyphenyl)-2-methylpentan-3-ol, and use thereof for the preparation of tapentadol or a pharmaceutically acceptable salt thereof. Disclosed further herein is an improved and industrially advantageous optical resolution method for resolving (2R,3R)/(2S,3S)-[3-(3-methoxyphenyl)-2-methylpentyl]-dimethylamine, and use thereof for the preparation of tapentadol or a pharmaceutically acceptable salt thereof. Disclosed also herein is an improved, commercially viable and industrially advantageous process for the preparation of tapentadol or a pharmaceutically acceptable salt thereof in high yield and purity.

### BACKGROUND

U.S. Pat. No. 6,248,737 reissued as USRE39593 discloses a variety of 1-phenyl-3-dimethylaminopropane compounds, processes for their preparation, pharmaceutical compositions comprising the compounds, and methods of use thereof. These compounds have the utility as analgesic active ingredients in pharmaceutical compositions. Among them, Tapentadol hydrochloride, 3-[(1R,2R)-3-(dimethylamino)-1-ethyl-2-methylpropyl]phenol hydrochloride, is a centrally-acting analgesic with a unique dual mode of action as an agonist at the g-opioid receptor and as a norepinephrine reuptake inhibitor. Tapentadol hydrochloride is represented by the following structural formula:

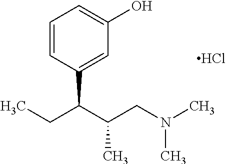 [SMILES: CC[C@@H](c1cccc(O)c1)[C@@H](C)CN(C)C ; Cl]

Various processes for the preparation of tapentadol, its enantiomers and related compounds, and their pharmaceutically acceptable salts are disclosed in U.S. Pat. Nos. USRE39593 and 6,344,558; and PCT Publication Nos. WO 2004/108658, WO 2005/000788, WO 2008/012046, WO 2008/012047 and WO 2008/012283.

As per the process exemplified in example 25 of the USRE39593 (hereinafter referred to as the '593 patent), (−)-(1R,2R)-3-(3-dimethylamino-1-ethyl-2-methylpropyl)-phenol hydrochloride is prepared by the reaction of (−)-(2S,3S)-1-dimethylamino-3-(3-methoxyphenyl)-2-methylpentan-3-ol hydrochloride with thionyl chloride to produce (−)-(2S,3S)-[3-chloro-3-(3-methoxyphenyl)-2-methylpentyl]-dimethylamine hydrochloride; followed by subsequent removal of the ‘Cl’ substituent by treatment with zinc borohydride, zinc cyanoborohydride or tin cyanoborohydride, to produce (−)-(2R,3R)-[3-(3-methoxyphenyl)-2-methylpentyl]-dimethylamine hydrochloride, which is then converted into (−)-(1R,2R)-3-(3-dimethylamino-1-ethyl-2-methylpropyl)-phenol hydrochloride by reaction with concentrated hydrobromic acid at reflux.

The synthesis of (2RS,3RS)-1-dimethylamino-3-(3-methoxyphenyl)-2-methylpentan-3-ol is described in the '593 patent. The separation of the diastereoisomers, that is the two enantiomeric pairs, is carried out by hydrochloride precipitation with trimethylchlorosilane/water in 2-butanone. The resolution of the racemic mixture of the two enantiomers of (2R,3R) and (2S,3S) configuration is carried by separation on a chiral HPLC column.

Methods involving column chromatographic separation of enantiomers on chiral stationary phases are generally undesirable for large-scale operations as they require additional expensive setup, adding to the cost of production, thereby making the process commercially unfeasible.

In the preparation of tapentadol, (−)-(2R,3R)-1-dimethylamino-3-(3-methoxyphenyl)-2-methylpentan-3-ol of formula III:

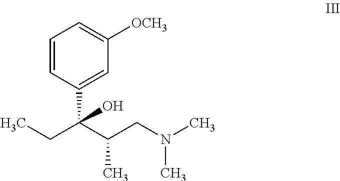 [SMILES: CC[C@](O)(c1cccc(OC)c1)[C@@H](C)CN(C)C]

is a key intermediate having the desired stereo chemistry.

U.S. Patent application No. 2008/0269524 discloses a resolution method for the separation of the two enantiomers from the enantiomeric pair, (2R,3R)/(2S,3S)-1-dimethylamino-3-(3-methoxyphenyl)-2-methylpentan-3-ol, with the aid of a chiral auxiliary, such as (+)-di-O,O′-p-toluoyltartaric acid, (−)-di-O,O′-p-toluoyltartaric acid and L-(+)-tartaric acid, in the presence of a suitable solvent such as 2-butanone.

U.S. Pat. No. 7,550,624 (hereinafter referred to as the '624 patent) discloses various pharmaceutically active salts and esters of 1-dimethylamino-3-(3-methoxyphenyl)-2-methylpentane-3-ol and 3-(3-dimethylamino-1-ethyl-1-hydroxy-2-methylpropyl)-phenol, and methods of using the same for treating or inhibiting increased urinary urgency or urinary incontinence and/or pain. The salts include ibuprofen, (S)-(+)-ibuprofen, (S)-(+)-naproxen, diclofenac, acetyl-salicylic acid, dipyron, indomethacin, ketoprofen, isoxicam, flurbiprofen, piroxicam and phenylbutazone. However, the '624 patent neither describes any resolution methods of the intermediates nor the use of any of the above salts for resolution processes.

The processes for the preparation of tapentadol or a pharmaceutically acceptable salt thereof and its intermediates, for example, (−)-(2R,3R)-1-dimethylamino-3-(3-methoxyphenyl)-2-methylpentan-3-ol of formula III, disclosed in the above mentioned prior art have the following disadvantages and limitations:
i) long reaction time periods, low yields and low purities of the products;
ii) the use of large volumes of solvents;
iii) the use of large volumes of concentrated hydrobromic acid (more than 23 volumes per 1 gm of the (−)-(2R,3R)-[3-(3-methoxyphenyl)-2-methylpentyl]-dimethylamine hydrochloride) in the demethylation reaction;
iv) the use of thionyl chloride for chlorination reaction, which is toxic and dangerous to the environment; and
v) the overall processes generate a large quantity of chemical waste which is difficult to treat.

Based on the aforementioned drawbacks, the prior art processes have been found to be unsuitable for the preparation of (−)-(2R,3R)-1-dimethylamino-3-(3-methoxyphenyl)-2-methylpentan-3-ol and tapentadol at lab scale and in commercial scale operations.

A need remains for an improved and commercially viable process of preparing (−)-(2R,3R)-1-dimethylamino-3-(3-methoxyphenyl)-2-methylpentan-3-ol of formula III with high yields and high enantiomeric purity, to resolve the problems associated with the processes described in the prior art, and that will be suitable for large-scale preparation. Desirable process properties include non-hazardous conditions, environmentally friendly and easy to handle reagents, reduced reaction time periods, reduced cost, greater simplicity, increased purity, and increased yield of the product, thereby enabling the production of tapentadol and its pharmaceutically acceptable acid addition salts in high purity and in high yield.

### SUMMARY

In one aspect, provided herein is an efficient, convenient, commercially viable and environmentally friendly resolution process for the preparation of enantiomerically pure tapentadol intermediate, (−)-(2R,3R)-1-dimethylamino-3-(3-methoxyphenyl)-2-methylpentan-3-ol of formula III, using S-naproxen as a chiral auxiliary, and a suitable solvent. The process avoids the tedious and cumbersome procedures of the prior art and is convenient to operate on a commercial scale.

In still another aspect, encompassed herein is the use of enantiomerically pure (−)-(2R,3R)-1-dimethylamino-3-(3-methoxyphenyl)-2-methylpentan-3-ol obtained by the process disclosed herein for preparing tapentadol or a pharmaceutically acceptable salt thereof.

In another aspect, provided herein is a resolution process for the preparation of enantiomerically pure tapentadol intermediate, (−)-(2R,3R)-[3-(3-methoxyphenyl)-2-methylpentyl]-dimethylamine.

In another aspect, the present disclosure provides a convenient, commercially viable and environmentally friendly process for the preparation of tapentadol or a pharmaceutically acceptable salt thereof. Moreover, the reagents used for process described herein are non-hazardous and easy to handle at commercial scale. In addition, the process requires a reduced reaction time compared to the prior art processes.

The resolution process for the preparation of (−)-(2R,3R)-1-dimethylamino-3-(3-methoxyphenyl)-2-methylpentan-3-ol of formula III using S-naproxen disclosed herein has the following advantages over the processes described in the prior art:
i) the yield and purity of the (−)-(2R,3R)-1-dimethylamino-3-(3-methoxyphenyl)-2-methylpentan-3-ol are increased;
ii) the resolution process is completed in shorter reaction time periods; and
iii) the resolving agent S-naproxen is easily recyclable and it can be used further for resolution processes.

The process for the preparation of tapentadol or a pharmaceutically acceptable salt thereof disclosed herein has the following advantages over the processes described in the prior art:
i) the amount of hydrobromic acid (2 to 4 volumes) used for the demethylation reaction is minimized with the modification of reaction conditions, thereby reducing the acidic waste produced;
ii) the overall process time is shortened;
iii) the process avoids the use of hazardous chemicals, and tedious and cumbersome procedures like HBr distillation at post reaction completion; iv) the process involves the use of reduced and more appropriate volumes of the solvents;
v) the process involves easy work-up methods and simple isolation processes;
vi) the process avoids the use of multiple purification/crystallization methods and column chromatographic purifications; and
vii) the overall yield and purities of the product are increased.

### DETAILED DESCRIPTION

In one aspect, there is provided a resolution process for the preparation of (−)-(2R,3R)-1-dimethylamino-3-(3-methoxyphenyl)-2-methylpentan-3-ol of formula III:

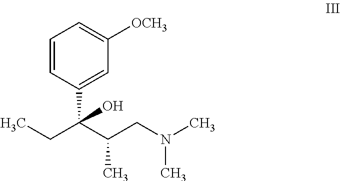 [SMILES: CC[C@](O)(c1cccc(OC)c1)[C@@H](C)CN(C)C]

or an acid addition salt thereof, comprising:
a) treating the enantiomeric pair (2R,3R)/(2S,3S)-1-dimethylamino-3-(3-methoxyphenyl)-2-methylpentan-3-ol or an acid addition salt thereof with (αS)-6-methoxy-α-methyl-2-naphthaleneacetic acid (S-naproxen) in a first solvent to produce a reaction mass containing the diastereomeric mixture of desired diastereomeric salt, (2R,3R)-1-dimethylamino-3-(3-methoxyphenyl)-2-methylpentan-3-ol S-naproxen salt, and undesired diastereomeric salt, (2S,3S)-1-dimethylamino-3-(3-methoxyphenyl)-2-methylpentan-3-ol S-naproxen salt;
b) separating the undesired diastereomeric salt from the diastereomeric mixture obtained in step-(a) to produce the desired diastereomeric salt; and
c) neutralizing the separated diastereomers of step-(b), specifically the desired diastereomeric salt, with a base in a second solvent to provide enantiomerically pure (−)-(2R,3R)-1-dimethylamino-3-(3-methoxyphenyl)-2-methylpentan-3-ol of formula III, and optionally converting the enantiomerically pure compound of formula III obtained into an acid addition salt thereof.

The term “enantiomerically pure compound of formula III” refers to the compound of formula III having enantiomeric purity greater than about 95%, specifically greater than about 98%, more specifically greater than about 99.5%, and most specifically greater than about 99.9% measured by HPLC.

Exemplary first solvents used in step-(a) include, but are not limited to, water, an alcohol, a ketone, a cyclic ether, an aliphatic ether, a hydrocarbon, a chlorinated hydrocarbon, a nitrile, an ester, a polar aprotic solvent, and mixtures thereof. The term solvent also includes mixtures of solvents.

In one embodiment, the first solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically, the first solvent is selected from the group consisting of water, methanol, ethanol, isopropanol, acetonitrile, and mixtures thereof; and most specifically isopropanol, acetonitrile, and mixtures thereof.

In another embodiment, the reaction in step-(a) is carried out at a temperature of −20° C. to the reflux temperature of the solvent used for at least 15 minutes, specifically at a temperature of about 0° C. to about 60° C. for about 30 minutes to about 8 hours, and more specifically at a temperature of about 20° C. to about 50° C. for about 2 hours to about 6 hours.

The separation of diastereomers in step-(b) may be required to provide stereomers with desired optical purity. It is well known that diastereomers differ in their properties such as solubility, and thus can be separated based on the differences in their properties. The separation of the diastereomers can be performed using the methods known to the person skilled in the art. These methods include chromatographic techniques and fractional crystallization, and a preferable method is fractional crystallization.

In one embodiment, a solution of the diastereomeric mixture is subjected to fractional crystallization. The solution of the diastereomeric mixture may be a solution of the reaction mixture obtained as above or a solution prepared by dissolving the isolated diastereomeric mixture in a solvent. Specific solvents used for the separation include, but are not limited to, water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically, the solvent is selected from the group consisting of water, methanol, ethanol, isopropanol, acetonitrile, and mixtures thereof; and most specifically isopropanol, acetonitrile, and mixtures thereof.

The fractional crystallization of one diastereomer from the solution of the diastereomeric mixture can be performed by conventional methods such as cooling, partial removal of solvents, using an anti-solvent, seeding, or a combination thereof.

Fractional crystallization can be repeated until the desired chiral purity is obtained. In general, usually one or two crystallizations may be sufficient.

In one embodiment, the separation in step-(b) is carried out by filtering the separated undesired diastereomeric salt and the resulting mother liquor, which contains the desired diastereomeric salt (2R,3R)-1-dimethylamino-3-(3-methoxyphenyl)-2-methylpentan-3-ol S-naproxen salt, is collected and then used in the next step for release of the base to produce the desired enantiomer (−)-(2R,3R)-1-dimethylamino-3-(3-methoxyphenyl)-2-methylpentan-3-ol of formula III.

In one embodiment, the base used in step-(c) is an organic or inorganic base. Specific organic bases are triethylamine, trimethylamine, dimethyl amine and tert-butyl amine.

In another embodiment, the base is an inorganic base. Exemplary inorganic bases include, but are not limited to, hydroxides, alkoxides, bicarbonates and carbonates of alkali or alkaline earth metals; and ammonia. Specific inorganic bases are aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide, and more specifically aqueous ammonia, sodium hydroxide, potassium hydroxide, sodium carbonate and potassium carbonate.

Exemplary second solvents used in step-(c) include, but are not limited to, water, an alcohol, a ketone, a cyclic ether, an aliphatic ether, a hydrocarbon, a chlorinated hydrocarbon, a nitrile, an ester, and mixtures thereof. The term solvent also includes mixtures of solvents.

In one embodiment, the second solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, and mixtures thereof; more specifically, the second solvent is selected from the group consisting of water, methylene chloride, n-hexane, n-heptane, cyclohexane, toluene, xylene, and mixtures thereof; and most specifically a mixture of water and methylene chloride.

In one embodiment, the pH of the reaction mass in step-(c) is adjusted to above 7, and specifically between 9 and 10.

The reaction mass containing the enantiomerically pure compound of formula III obtained in step-(c) may be subjected to usual work up such as a washing, a filtration, an extraction, an evaporation, or a combination thereof.

In one embodiment, the enantiomerically pure (−)-(2R,3R)-1-dimethylamino-3-(3-methoxyphenyl)-2-methylpentan-3-ol of formula III formed in step-(c) is isolated from a suitable organic solvent by methods such as cooling, seeding, partial removal of the solvent from the solution, by adding an anti-solvent to the solution, evaporation, vacuum distillation, or a combination thereof.

In another embodiment, the acid addition salt of (−)-(2R,3R)-1-dimethylamino-3-(3-methoxyphenyl)-2-methylpentan-3-ol is derived from a therapeutically acceptable acid such as hydrochloric acid, acetic acid, propionic acid, sulfuric acid and nitric acid. A specific salt is (−)-(2R,3R)-1-dimethylamino-3-(3-methoxyphenyl)-2-methylpentan-3-ol hydrochloride.

In another embodiment, the resolution procedure disclosed herein can be used to resolve mixtures that comprise both enantiomers of (2R,3R)/(2S,3S)-1-dimethylamino-3-(3-methoxyphenyl)-2-methylpentan-3-ol in any proportion. Therefore, this procedure is applicable both to perform the optical resolution of a racemic mixture of (2R,3R)/(2S,3S)-1-dimethylamino-3-(3-methoxyphenyl)-2-methylpentan-3-ol (that is to say, that in which the two enantiomers are present in a 1:1 ratio) and for the optical resolution of non-racemic mixtures of (2R,3R)/(2S,3S)-1-dimethylamino-3-(3-methoxyphenyl)-2-methylpentan-3-ol (in which one of the enantiomers is present in greater proportion), obtained by any physical or chemical method.

Tapentadol and pharmaceutically acceptable salts of tapentadol can be prepared in high purity by using the enantiomerically pure (−)-(2R,3R)-1-dimethylamino-3-(3-methoxyphenyl)-2-methylpentan-3-ol of formula III or its acid addition salts thereof obtained by the methods disclosed herein, by known methods.

In one embodiment, the processes disclosed herein are adapted to the preparation of tapentadol or a pharmaceutically acceptable salt thereof in high enantiomeric and chemical purity.

According to another aspect, there is provided a resolution process for the preparation of (−)-(2R,3R)-[3-(3-methoxyphenyl)-2-methylpentyl]-dimethylamine of formula II:

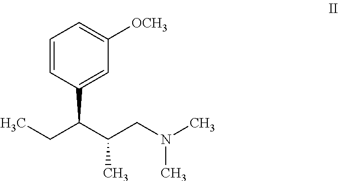 [SMILES: CC[C@@H](c1cccc(OC)c1)[C@@H](C)CN(C)C]

or an acid addition salt thereof, comprising:
a) treating the enantiomeric pair (2R,3R)/(2S,3S)-[3-(3-methoxyphenyl)-2-methylpentyl]-dimethylamine or an acid addition salt thereof with a suitable optically active acid in a first solvent to produce a reaction mass containing the diastereomeric mixture;
b) separating the desired diastereomeric salt from the diastereomeric mixture obtained in step-(a); and
c) neutralizing the desired diastereomeric salt obtained in step-(b) with a base in a second solvent to produce enantiomerically pure (−)-(2R,3R)-[3-(3-methoxyphenyl)-2-methylpentyl]-dimethylamine of formula II, and optionally converting the enantiomerically pure compound of formula II obtained into an acid addition salt thereof.

The term “enantiomerically pure compound of formula II” refers to the compound of formula II having enantiomeric purity greater than about 95%, specifically greater than about 98%, more specifically greater than about 99.5%, and most specifically greater than about 99.9% measured by HPLC.

Exemplary optically active acids used in step-(a) include, but are not limited to, optically active di-aroyl-tartaric acid, S-naproxen, malic acid, mandelic acid and its derivatives, and camphorsulphonic acid and its derivatives.

In one embodiment, the optically active acid is selected from the group consisting of S-naproxen, (−)-di-p-toluoyl-L-tartaric acid, (+)-di-p-toluoyl-D-tartaric acid, (−)-dibenzoyl-L-tartaric acid, (+)-dibenzoyl-D-tartaric acid, and hydrates thereof. Most specific optically active acids are (−)-di-p-toluoyl-L-tartaric acid and S-naproxen.

The optically active acid in step-(a) can be optionally used as a mixture with other acids (adjuvant acids) that can be organic or inorganic acids, such as hydrochloric acid, p-toluensulphonic acid, methanosulphonic acid or a mixture thereof, in molar proportions that vary between 0.5% and 50% (this molar percentage refers to the total of the mixture of the chiral acid and the adjuvant acid).

Exemplary first solvents used in step-(a) include, but are not limited to, water, an alcohol, a ketone, a cyclic ether, an aliphatic ether, a hydrocarbon, a chlorinated hydrocarbon, a nitrile, an ester, a polar aprotic solvent, and mixtures thereof.

In one embodiment, the first solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof more specifically, the first solvent is selected from the group consisting of water, methanol, ethanol, isopropanol, acetonitrile, and mixtures thereof; and most specifically a mixture of water and methanol.

In another embodiment, the reaction in step-(a) is carried out at a temperature of −20° C. to the reflux temperature of the solvent used for at least 30 minutes, specifically at a temperature of about 0° C. to about 80° C. for about 30 minutes to about 15 hours, and more specifically at a temperature of about 20° C. to about 75° C. for about 3 hours to about 10 hours.

In one embodiment, the separation of diastereomers in step-(b) is carried out by fractional crystallization.

Preferably, a solution of the diastereomeric mixture is subjected to fractional crystallization. The solution of the diastereomeric mixture may be a solution of the reaction mixture obtained as above or a solution prepared by dissolving the isolated diastereomeric mixture in a solvent. The solvent used for the separation is selected from the group as described above.

The fractional crystallization of one diastereomer from the solution of the diastereomeric mixture can be performed by conventional methods such as cooling, partial removal of solvents, using an anti-solvent, seeding or a combination thereof.

In one embodiment, the base used in step-(c) is an organic or inorganic base selected from the group as described above. Specific inorganic bases are aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide, and more specifically aqueous ammonia, sodium hydroxide, potassium hydroxide, sodium carbonate and potassium carbonate.

In another embodiment, the second solvent used in step-(c) is selected from the group as described above. Specific second solvents are water, methylene chloride, n-hexane, n-heptane, cyclohexane, toluene, xylene, and mixtures thereof; and most specifically a mixture of water and methylene chloride.

In one embodiment, the pH of the reaction mass in step-(c) is adjusted to above 7, and specifically between 7 and 8.

The reaction mass containing the enantiomerically pure compound of formula II obtained in step-(c) may be subjected to usual work up such as a washing, a filtration, an extraction, an evaporation, or a combination thereof.

In one embodiment, the enantiomerically pure (−)-(2R,3R)-[3-(3-methoxyphenyl)-2-methylpentyl]-dimethylamine of formula II formed in step-(c) is isolated from a suitable organic solvent by methods such as cooling, seeding, partial removal of the solvent from the solution, by adding an anti-solvent to the solution, evaporation, vacuum distillation, or a combination thereof.

In another embodiment, the acid addition salt of (−)-(2R,3R)-[3-(3-methoxyphenyl)-2-methylpentyl]-dimethylamine is derived from a therapeutically acceptable acid such as hydrochloric acid, acetic acid, propionic acid, sulfuric acid and nitric acid. A specific salt is (−)-(2R,3R)-[3-(3-methoxyphenyl)-2-methylpentyl]-dimethylamine hydrochloride.

Tapentadol and pharmaceutically acceptable salts of tapentadol can be prepared in high purity by using the enantiomerically pure (−)-(2R,3R)-[3-(3-methoxyphenyl)-2-methylpentyl]-dimethylamine of formula II or its acid addition salts thereof obtained by the methods disclosed herein, by known methods.

According to another aspect, there is provided a process for preparing tapentadol, 3-[(1R,2R)-3-(dimethylamino)-1-ethyl-2-methylpropyl]phenol, of formula I:

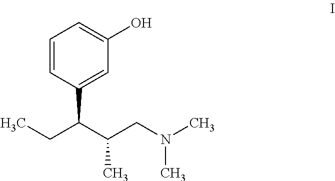 [SMILES: CC[C@@H](c1cccc(O)c1)[C@@H](C)CN(C)C]

or a pharmaceutically acceptable salt thereof, comprising
a) reacting the enantiomeric pair (2R,3R)/(2S,3S)-1-dimethylamino-3-(3-methoxyphenyl)-2-methylpentan-3-ol or an acid addition salt thereof with trifluoroacetic anhydride in a first solvent to produce a reaction mass;
b) hydrogenating the reaction mass obtained in step-(a) in the presence of a hydrogenation catalyst to produce the enantiomeric pair (2R,3R)/(2S,3S)-[3-(3-methoxyphenyl)-2-methylpentyl]-dimethylamine or an acid addition salt thereof;
c) resolving the enantiomeric pair obtained in step-(b) with a suitable optically active acid to produce an enantiomerically pure (−)-(2R,3R)-[3-(3-methoxyphenyl)-2-methylpentyl]-dimethylamine of formula II or an acid addition salt thereof, wherein the optically active acid is selected from the group consisting of optically active di-aroyl-tartaric acid, S-naproxen, malic acid, mandelic acid and its derivatives, and camphorsulphonic acid and its derivatives;
d) demethylating the enantiomerically pure compound of formula II obtained in step-(c) using a demethylating agent in a second solvent to produce tapentadol of formula I, and optionally converting the tapentadol of formula I obtained into a pharmaceutically acceptable salt thereof; and
e) optionally, purifying the tapentadol or a pharmaceutically acceptable salt thereof obtained in step-(d) using a third solvent to produce highly pure tapentadol or a pharmaceutically acceptable salt thereof.

Exemplary first solvents used in step-(a) include, but are not limited to, water, an alcohol, a ketone, a cyclic ether, an aliphatic ether, a hydrocarbon, a chlorinated hydrocarbon, a nitrile solvent, and mixtures thereof.

In one embodiment, the first solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, dichloromethane, dichloroethane, chloroform, carbon tetrachloride, tetrahydrofuran, 2-methyl tetrahydrofuran, dioxane, diethyl ether, diisopropyl ether, monoglyme, diglyme, n-pentane, n-hexane, n-heptane, cyclohexane, toluene, xylene, and mixtures thereof; more specifically, the first solvent is selected from the group consisting of methanol, ethanol, isopropanol, acetonitrile, tetrahydrofuran, 2-methyl tetrahydrofuran, and mixtures thereof; and most specifically 2-methyl tetrahydrofuran.

In another embodiment, the reaction in step-(a) is carried out at a temperature of −20° C. to 50° C. for at least 10 minutes, specifically at a temperature of about 0° C. to about 40° C. for about 20 minutes to about 6 hours, and more specifically at a temperature of about 0° C. to about 30° C. for about 1 hour to about 4 hours.

Exemplary hydrogenation catalysts used in step-(b) include, but are not limited to, palladium hydroxide, palladium on carbon, platinum on carbon, platinum oxide, rhodium on carbon, rhodium on alumina. A specific hydrogenation catalyst is palladium on carbon.

In one embodiment, the hydrogenation reaction in step-(b) is carried out at a temperature of 0° C. to the reflux temperature of the solvent used for at least 30 minutes, specifically at a temperature of about 0° C. to about 50° C. for about 1 hour to about 7 hours, and more specifically at about 20° C. to about 45° C. for about 2 hours to about 5 hours.

In one embodiment, the hydrogenation reaction in step-(b) is carried out under hydrogen pressure or in the presence of hydrogen transfer reagent.

Exemplary hydrogen transfer reagent include, but are not limited to, formic acid, salts of formic acid such as ammonium formate, sodium formate, trialkyl ammonium formates, hydrazine, 1,3-cyclohexadiene, 1,4-cyclohexadiene and cyclohexene.

As used herein, the term ‘alkyl’ means saturated, acyclic groups which may be straight or branched containing from one to about seven carbon atoms as exemplified by methyl, ethyl, propyl, isopropyl, butyl, hexyl or heptyl.

Specific hydrogen transfer reagents are formic acid, ammonium formate, sodium formate, trimethylammonium formate and tributylammonium formate; and more specifically ammonium formate.

In another embodiment, the hydrogenation catalyst is used in the ratio of about 0.05% (w/w) to 10% (w/w), specifically about 0.5% (w/w) to 2.5% (w/w), with respect to the enantiomeric pair (2R,3R)/(2S,3S)-1-dimethylamino-3-(3-methoxyphenyl)-2-methylpentan-3-ol in order to ensure a proper course of the reaction.

The reaction mass containing the enantiomeric pair (2R,3R)/(2S,3S)-[3-(3-methoxyphenyl)-2-methylpentyl]-dimethylamine or an acid addition salt obtained in step-(b) may be subjected to usual work up such as a washing, a filtration, an extraction, a pH adjustment, an evaporation, or a combination thereof. The reaction mass may be used directly in the next step to produce enantiomerically pure (−)-(2R,3R)-[3-(3-methoxyphenyl)-2-methylpentyl]-dimethylamine of formula II, or the enantiomeric pair (2R,3R)/(2S,3S)-[3-(3-methoxyphenyl)-2-methylpentyl]-dimethylamine may be isolated and then used in the next step.

In one embodiment, the enantiomeric pair (2R,3R)/(2S,3S)-[3-(3-methoxyphenyl)-2-methylpentyl]-dimethylamine is isolated from a suitable solvent by conventional methods such as cooling, seeding, partial removal of the solvent from the solution, by adding an anti-solvent to the solution, evaporation, vacuum distillation, or a combination thereof.

In another embodiment, the solvent used to isolate the enantiomeric pair (2R,3R)/(2S,3S)-[3-(3-methoxyphenyl)-2-methylpentyl]-dimethylamine is selected from the group consisting of water, an aliphatic ether, a hydrocarbon solvent, a chlorinated hydrocarbon, and mixtures thereof. Specifically, the solvent is selected from the group consisting of water, dichloromethane, diethyl ether, diisopropyl ether, n-heptane, n-pentane, n-hexane, cyclohexane, and mixtures thereof. A most specific solvent is dichloromethane.

In one embodiment, the resolution in step-(c) is carried out by the methods as described hereinabove.

Exemplary demethylating agents used in step-(d) include, but are not limited to, hydrobromic acid, aluminum chloride/thiourea, aluminium triiodide/tetrabutylammonium iodide and ClBH₂.Me₂S. A most specific demethylating agent is hydrobromic acid.

In one embodiment, the hydrobromic acid is used in the molar ratio of about 2 to 10 volumes, specifically about 3 to 4 volumes, per 1 gm of the (−)-(2R,3R)-[3-(3-methoxyphenyl)-2-methylpentyl]-dimethylamine of formula II in order to ensure a proper course of the reaction.

Exemplary second solvents used in step-(d) include, but are not limited to, water, an alcohol, a ketone, a cyclic ether, an aliphatic ether, a hydrocarbon, a chlorinated hydrocarbon, a nitrile solvent, and mixtures thereof. The term solvent also includes mixtures of solvents.

In one embodiment, the second solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, dichloromethane, dichloroethane, chloroform, carbon tetrachloride, tetrahydrofuran, 2-methyl tetrahydrofuran, dioxane, diethyl ether, diisopropyl ether, monoglyme, diglyme, n-pentane, n-hexane, n-heptane, cyclohexane, toluene, xylene, and mixtures thereof; and a and most specific second solvent is toluene.

In another embodiment, the reaction in step-(d) is carried out at a temperature of 0° C. to the reflux temperature of the solvent used for at least 20 minutes, specifically at a temperature of about 50° C. to about 120° C. for about 30 minutes to about 8 hours, and more specifically at a temperature of about 90° C. to about 115° C. for about 1 hour to about 4 hours.

The reaction mass containing the tapentadol obtained in step-(d) may be subjected to usual work up such as a washing, a filtration, an extraction, an evaporation, a pH adjustment, or a combination thereof.

In one embodiment, the tapentadol of formula I formed in step-(d) is isolated from a suitable solvent by the methods as described above.

Pharmaceutically acceptable salts of tapentadol can be prepared in high purity by using the highly pure tapentadol obtained by the method disclosed herein, by known methods.

Specific pharmaceutically acceptable salts of tapentadol include, but are not limited to, hydrochloride, hydrobromide, oxalate, nitrate, sulphate, phosphate, fumarate, succinate, maleate, besylate, tosylate, palmitate and tartrate; and more specifically hydrochloride.

Exemplary third solvents used in step-(e) include, but are not limited to, water, an alcohol, a ketone, and mixtures thereof. The term solvent also includes mixtures of solvents.

In one embodiment, the third solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, and mixtures thereof; and a and most specific third solvent is acetone or methyl ethyl ketone.

In another embodiment, the purification in step-(e) is carried out by a process comprising providing a solution of tapentadol or a pharmaceutically acceptable salt thereof in the third solvent, optionally, subjecting the solution to carbon treatment or silica gel treatment; and isolating the highly pure of tapentadol or a pharmaceutically acceptable salt thereof from the solution by the methods as described above.

The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate by removing charcoal or silica gel. Specifically, the finely powdered carbon is an active carbon. A specific mesh size of silica gel is 40-500 mesh, and more specifically 60-120 mesh.

The highly pure tapentadol or a pharmaceutically acceptable salt thereof obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or a reduced pressure, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

In another embodiment, the highly pure tapentadol or a pharmaceutically acceptable salt thereof disclosed herein has a total purity of greater than about 99%, specifically greater than about 99.5%, more specifically greater than about 99.9%, and most specifically greater than about 99.95% as measured by HPLC. For example, the purity of the highly pure tapentadol or a pharmaceutically acceptable salt thereof is about 99% to about 99.9%, or about 99.5% to about 99.99%.

The following examples are given for the purpose of illustrating the present disclosure and should not be considered as limitation on the scope or spirit of the disclosure.

### EXAMPLES

### Example 1

### Step-I: Resolution of the racemic mixture of (2R,3R)/(2S,3S)-1-Dimethylamino-3-(3-methoxyphenyl)-2-methylpentan-3-ol using S-Naproxen

### Method A:

Acetonitrile (5 ml) and S-naproxen (0.91 g) were added to the racemic mixture of (2R,3R)/(2S,3S)-1-(dimethylamino)-3-(3-methoxyphenyl)-2-methylpentan-3-ol (1g). The resulting mixture was heated to 40-45° C. and then stirred for 3 hours at 25-30° C. The resulting mass was filtered, then the separated white solid was washed with acetonitrile (1 ml) and then dried at 40-45° C. to produce 0.99 g of undesired diastereomeric salt of (2S,3S)-1-dimethylamino-3-(3-methoxyphenyl)-2-methylpentan-3-ol with S-naproxen. The resulting mother liquor, which contains the desired diastereomeric salt of (2R,3R)-1-dimethylamino-3-(3-methoxyphenyl)-2-methylpentan-3-ol with S-naproxen, was collected and further used for release of the base to produce the desired enantiomer.

### Method B:

Isopropyl alcohol (5 ml) and S-naproxen (0.91 g) were added to the racemic mixture of (2R,3R)/(2S,3S)-1-(dimethylamino)-3-(3-methoxyphenyl)-2-methylpentan-3-ol (1g), the resulting mixture was heated to 40-45° C. and then stirred for 3 hours at 25-30° C. The resulting mass was filtered, the separated white solid was washed with isopropyl alcohol (1 ml) and then dried at 40-45° C. to produce 0.99 g of undesired diastereomeric salt of (2S,3S)-1-dimethylamino-3-(3-methoxyphenyl)-2-methylpentan-3-ol with S-naproxen. The resulting mother liquor, which contains the desired diastereomeric salt of (2R,3R)-1-dimethylamino-3-(3-methoxyphenyl)-2-methylpentan-3-ol with S-naproxen, was collected and further used for release of the base to produce the desired enantiomer.

### Step-II: Isolation of (−)-(2R,3R)-1-dimethylamino-3-(3-methoxyphenyl)-2-methylpentan-3-ol

(desired enantiomer) from the Mother Liquors obtained in step-I The mother liquors obtained in method-A and method-B of step-I were combined and then distilled under vacuum at 20-25° C. to dryness. Water (20 ml) and dichloromethane (40 ml) were added to the resulting residue, followed by adjusting the pH to 9-10 using a 20% sodium hydroxide solution. The resulting mixture was stirred for 20 minutes at 20-25° C., followed by separation of the layers. The separated organic layer was dried over anhydrous sodium sulfate and then distilled under vacuum at 40° C. to produce 1.1 g of (−)-(2R,3R)-1-dimethylamino-3-(3-methoxyphenyl)-2-methylpentan-3-ol as an oily mass [Specific optical rotation (SOR)=−17.4° at 20° C., C=1, methanol].

### Step-III: Isolation of (+)-(2S,3S)-1-dimethylamino-3-(3-methoxyphenyl)-2-methylpentan-3-ol (undesired enantiomer)

### Method A:

Water (10 ml) and dichloromethane (20 ml) were added to the filtered solid (0.9 g, undesired diastereomeric salt) obtained in method-A of step-I, followed by adjusting the pH of the resulting mixture to 9-10 using 20% sodium hydroxide solution. The resulting mixture was stirred for 20 minutes at 20-25° C., followed by separation of the layers. The separated organic layer was dried over anhydrous sodium sulfate and then distilled under vacuum at 40° C. to produce 0.41 g of (+)-(2S,3S)-1-dimethylamino-3-(3-methoxyphenyl)-2-methylpentan-3-ol as an oily mass [Specific optical rotation (SOR)=+18.5° at 20° C., C=1, methanol].

### Method B:

Water (10 ml) and dichloromethane (20 ml) were added to the filtered solid (0.9 g, undesired diastereomeric salt) obtained in method-B of step-I, followed by adjusting the pH of the resulting mixture to 9-10 using 20% sodium hydroxide solution. The resulting mixture was stirred for 20 minutes at 20-25° C., followed by separation of the layers. The separated organic layer was dried over anhydrous sodium sulfate and then distilled under vacuum at 40° C. to produce 0.41 g of (+)-(2S,3S)-1-dimethylamino-3-(3-methoxyphenyl)-2-methylpentan-3-ol as an oily mass [Specific optical rotation (SOR)=+18.8° at 20° C., C=1, methanol].

### Example 2

### Preparation of 1-Dimethylamino-2-methylpentan-3-one

3-Pentanone (2000 g), dimethyl ammonium chloride (978 g), 36% formaldehyde (968 g) and 36% hydrochloric acid (40 ml) were taken into a reaction flask and the mixture was heated at 85° C. under stirring for 15 hours. The reaction mass was cooled to 20±2° C., the stirring was stopped, followed by separation of the organic layer. The resulting aqueous layer was placed in a reaction flask, followed by the addition of diisopropyl ether (500 ml), and stirring for 30 minutes. The above solution was transferred into the separating funnel, and the bottom aqueous layer was separated and transferred into the same reaction flask. The aqueous layer was cooled to 15-20° C., followed by the addition of 30% sodium hydroxide solution to adjust pH to 12-13 (aprox. 1800 ml). The resulting mass was stirred for 30 minutes and the solution was transferred into the separating funnel. The upper organic layer was collected in a dry container. The bottom aqueous layer was separated and transferred into the same flask, followed by the addition of dichloromethane (1000 ml). The resulting mixture was stirred for 30 minutes and the solution was transferred into the separating funnel. The bottom organic layer was separated. The organic layers were combined and then dried over sodium sulphate, followed by distillation of dichloromethane at 45-50° C. The resulting oily mass was cooled to 20-25° C. and then subjected to high vacuum distillation to produce 1340 g of 1-dimethylamino-2-methylpentan-3-one as an oily mass (Purity by GC: 92.71%).

### Example 3

### Preparation of racemic mixture of (2R,3R)/(2S,3S)-1-Dimethylamino-3-(3-methoxyphenyl)-2-methylpentan-3-ol hydrochloride

Tetrahydrofuran (400 ml) and magnesium metal (64 g) were taken under nitrogen atmosphere, followed by the addition of one crystal of iodine to initiate reaction, and then heating the mixture at 62±2° C. To the resulting mass was added a mixture of 3-bromo anisole (450 g) and tetrahydrofuran (800 ml) while maintaining the temperature at 67-70° C. The reaction mass obtained, after complete addition of the above mixture, was refluxed for 90 minutes and then cooled to 0° C. A solution of 1-dimethylamino-2-methylpentan-3-one (250 g) in tetrahydrofuran (800 ml) was added to the above mass at 0-10° C. The ice was removed after completion of the addition, followed by stirring the reaction mass at 20-25° C. for 15 hours and further cooling to 10° C. A solution of ammonium chloride (20%, 600 ml) was added to the reaction mass at below 20° C. and maintained the reaction mass at 20-25° C. under stirring for 30 minutes. The resulting mass was followed by the addition of dichloromethane (600 ml) and stirring the mass for 10-15 minutes. The reaction mass was transferred into a separating funnel and allowed it to settle for 10-15 minutes, followed by the separation of the upper organic layer. The resulting aqueous layer was separated and then placed into a flask, followed by the addition of water (600 ml) and dichloromethane (600 ml) and adjusting the pH of the resulting mixture to 7-8 using acetic acid at 20-25° C. The reaction mixture was stirred for 10-15 minutes, followed by transferring into the separating funnel and allowing it settle for 10-15 minutes. The upper organic layer was separated, followed by combining the total organic layer and washing with water (2×500 ml). The resulting organic layer was dried over anhydrous sodium sulphate (25 g), followed by distillation of dichloromethane completely at 40±2° C. and further applying high vacuum at 40±2° C. for 30 minutes in order to distil out the dichloromethane completely. Acetone (800 ml) was added to the oily mass, followed by cooling to 0° C. and bubbling hydrogen chloride gas at 0-10° C. to adjust the pH to below 1. The resulting mass was stirred for 4-5 hours at 0-5° C. The separated solid was filtered, washed with pre-cooled acetone (50 ml) and then the material was dried at 40-45° C. under vacuum till constant weight to produce 168 g of racemic mixture of (2R,3R)/(2S,3S)-1-dimethylamino-3-(3-methoxyphenyl)-2-methylpentan-3-ol hydrochloride (Purity by HPLC: 99.55%).

### Example 4

### Preparation of (2R,3R)/(2S,3S)-[3-(3-Methoxyphenyl)-2-methylpentyl]-dimethylamine

(2R,3R)/(2S,3S)-1-Dimethylamino-3-(3-methoxyphenyl)-2-methylpentan-3-ol (100 g) and 2-methyltetrahydrofuran (250 ml) were taken into a reaction flask, the mixture was cooled to 0° C., followed by the addition of trifluoro acetic anhydride (95 g) at 0-10° C. The reaction mixture was stirred for 10 minutes at 0-10° C., the ice bath was removed, and the resulting mass was stirred for 2 hours at 20-25° C. The reaction mass was transferred into an autoclave, the flask was washed with 2-methyltetrahydrofuran (50 ml), and then transferred into the autoclave, followed by the addition of Pd/C (10 g) and then applying hydrogen pressure (3-4 Kg). The resulting mass was heated at 40° C. and then stirred for 2-3 hours at 40-45° C. under pressure. The reaction mass was cooled to 20-25° C., followed by filtration through a hyflo bed and removing the solvent completely by distillation. Water (200 ml) and dichloromethane (400 ml) were added to the resulting mass. Sodium hydroxide solution (20%, 200 ml) was added to the resulting mixture to adjust the pH to 7.5-8.0, followed by stirring the reaction mass for 30 minutes. The bottom organic layer was separated and the aqueous layer was transferred into the same RB flask. Dichloromethane (200 ml) was added to the RB flask through a funnel and the resulting mixture was stirred for 10-15 minutes. The bottom organic layer was separated and combined with the first organic layer. The combined organic layer was washed with water (200 ml) and then dried over anhydrous sodium sulphate (25 g). The resulting mass was distilled under atmospheric pressure at 45-50° C. to remove dichloromethane, followed by complete removal of dichloromethane by high vacuum distillation at 40±2° C. for 1 hour to produce 90 g of (2R,3R)/(2S,3S)-[3-(3-methoxyphenyl)-2-methylpentyl]-dimethylamine as an oily mass (Purity by HPLC: 98.72%).

### Example 5

### Resolution of the racemic mixture of (2R,3R)/(2S,3S)-[3-(3-Methoxyphenyl)-2-methylpentyl]-dimethylamine using (−)-di-p-toluoyl-L-tartaric acid in aqueous methanol

10% Aqueous methanol (1500 ml) was taken into a RB flask, followed by the addition of (−)-di-p-toluoyl-L-tartaric acid (160 g) and stirring for 10-15 minutes. (2R,3R)/(2S,3S)-[3-(3-methoxyphenyl)-2-methylpentyl]-dimethylamine was added to the resulting mixture, followed by maintaining at 25±5° C. under stirring for 1 hour. The reaction mass was heated at 70±2° C. and then stirred for 15-20 minutes at 70±2° C., followed by cooling the mass to 22±2° C. for 3-4 hours. The resulting mass was stirred for 12 hours at 22±2° C., followed by filtration through the Buchner flask and washing the solid with methanol (50 ml). The product was suction dried for 30 minutes using vacuum, followed by drying under vacuum at 40±5° C. in a vacuum oven for 4 hours. The resulting chiral salt (118 g) and water (500 ml) were taken in a flask, followed by portion-wise addition of aqueous ammonia (200 ml) to adjust the pH to 7.5 to 8.0. Dichloromethane (500 ml) was added to the reaction mass under stirring, the resulting mass was stirred for 1 hour and the bottom organic layer was separated. The aqueous layer was extracted with dichloromethane (300 ml). The organic layers were combined, washed with water (2×150 ml) and then dried over anhydrous sodium sulphate (10 g). The resulting mass was filtered, followed by distillation of dichloromethane at 45-50° C. under atmospheric pressure. The resulting oily mass was again distilled under high vacuum at 50±2° C. for 1 hour for complete removal of dichloromethane to produce 42 g of (−)-(2R,3R)-[3-(3-methoxyphenyl)-2-methylpentyl]-dimethylamine as an oily mass (Purity by HPLC: 99.55%).

[SOR of Salt=(−)-103.9° at 20° C., c=1, methanol].

### Example 6

### Resolution of the racemic mixture of (2R,3R)/(2S,3S)-[3-(3-Methoxyphenyl)-2-methylpentyl]-dimethylamine using (−)-di-p-toluoyl-L-tartaric acid in methanol

(−)-Di-p-toluoyl-L-tartaric acid (3.2 g) was added to methanol (10 ml), the mixture was stirred for 10-15 minutes, followed by the addition of (2R,3R)/(2S,3S)-[3-(3-methoxyphenyl)-2-methylpentyl]-dimethylamine (2 g) and methanol (10 ml). The resulting mixture was heated at 45±5° C. and stirred for 15-20 minutes at 45±5° C. The reaction mass was cooled to 22±2° C. over a period of 3-4 hours and then stirred for 4 to 5 hours at 22±2° C. The resulting mass was filtered through Buchner flask and the solid was washed with 4 ml of methanol, followed by suction drying the product for 30 minutes using vacuum and then drying the product at 40±5° C. in a vacuum oven under vacuum for 4 hours to produce 2.5 g of the desired diastereomeric salt [SOR=(−)-100.2° at 20° C., c=1 methanol].

### Example 7

### Resolution of the racemic mixture of (2R,3R)/(2S,3S)-[3-(3-Methoxyphenyl)-2-methylpentyl]-dimethylamine using (−)-di-p-toluoyl-L-tartaric acid in acetone

(−)-Di-p-toluoyl-L-tartaric acid (3.2 g) was added to acetone (10 ml), the mixture was stirred for 10-15 minutes, followed by the addition of (2R,3R)/(2S,3S)-[3-(3-methoxyphenyl)-2-methylpentyl]-dimethylamine (2 g). To the resulting mixture was added acetone (20 ml), followed by heating the mass at 45±5° C. and stirring for 15-20 minutes at 45±5° C. The reaction mass was cooled to 22±2° C. over a period of 3-4 hours and then stirred for 4 to 5 hours at 22±2° C. The resulting mass was filtered through Buchner flask and the solid was washed with 2 ml of acetone, followed by suction drying the product for 30 minutes using vacuum and then drying the product at 40±5° C. in a vacuum oven under vacuum for 4 hours to produce 2.5 g of the desired diastereomeric salt [SOR=(−)-90.6° at 20° C., c=1 methanol].

### Example 8

### Resolution of the racemic mixture of (2R,3R)/(2S,3S)-[3-(3-Methoxyphenyl)-2-methylpentyl]-dimethylamine using (−)-di-p-toluoyl-L-tartaric acid in acetonitrile

(−)-Di-p-toluoyl-L-tartaric acid (3.2 g) was added to acetonitrile (10 ml), the mixture was stirred for 10-15 minutes, followed by the addition of (2R,3R)/(2S,3S)-[3-(3-methoxyphenyl)-2-methylpentyl]-dimethylamine (2 g). To the resulting mixture was added acetonitrile (10 ml), followed by heating the mass at 45±5° C. and stirring for 15-20 minutes at 45±5° C. The reaction mass was cooled to 22±2° C. over a period of 3-4 hours and then stirred for 4 to 5 hours at 22±2° C. The resulting mass was filtered through Buchner flask and the solid was washed with 4 ml of acetonitrile, followed by suction drying the product for 30 minutes using vacuum and then drying the product at 40±5° C. in a vacuum oven under vacuum for 4 hours to produce 2.1 g of the desired diastereomeric salt [SOR=(−)-94° at 20° C., c=1 methanol].

### Example 9

### Resolution of the racemic mixture of (2R,3R)/(2S,3S)-[3-(3-Methoxyphenyl)-2-methylpentyl]-dimethylamine using (−)-dibenzoyl-L-tartaric acid monohydrate in methanol

(−)-Dibenzoyl-L-tartaric acid monohydrate (3 g) was added to methanol (10 ml), the mixture was stirred for 10-15 minutes, followed by the addition of (2R,3R)/(2S,3S)-[3-(3-methoxyphenyl)-2-methylpentyl]-dimethylamine (2 g). The resulting mixture was stirred for 4 hours at 25±5° C. The resulting mass was filtered through Buchner flask and the solid was washed with 4 ml of methanol, followed by suction drying the product for 30 minutes using vacuum and then drying the product at 40±5° C. in a vacuum oven under vacuum for 4 hours.

### Example 10

### Resolution of the racemic mixture of (2R,3R)/(2S,3S)-[3-(3-Methoxyphenyl)-2-methylpentyl]-dimethylamine using (−)-Dibenzoyl-L-tartaric acid monohydrate in acetonitrile

(−)-Dibenzoyl-L-tartaric acid monohydrate (3 g) was added to acetonitrile (10 ml), the mixture was stirred for 10-15 minutes, followed by the addition of (2R,3R)/(2S,3S)-[3-(3-methoxyphenyl)-2-methylpentyl]-dimethylamine (2 g). To the resulting mixture was added acetonitrile (10 ml), followed by heating the mass at 45±5° C. and stirring for 15-20 minutes at 45±5° C. The reaction mass was cooled to 22±2° C. over a period of 3-4 hours and then stirred for 4 to 5 hours at 22±2° C. The resulting mass was filtered through Buchner flask and the solid was washed with 10 ml of acetonitrile, followed by suction drying the product for 30 minutes using vacuum, and then drying the product at 40±5° C. in a vacuum oven under vacuum for 4 hours.

### Example 11

### Resolution of the racemic mixture of (2R,3R)/(2S,3S)-[3-(3-Methoxyphenyl)-2-methylpentyl]-dimethylamine using (−)-Dibenzoyl-L-tartaric acid monohydrate in acetone

(−)-Dibenzoyl-L-tartaric acid monohydrate (3 g) was added to acetone (10 ml), the mixture was stirred for 10-15 minutes, followed by the addition of (2R,3R)/(2S,3S)-[3-(3-methoxyphenyl)-2-methylpentyl]-dimethylamine (2 g). To the resulting mixture was added acetone (20 ml), followed by heating the mass at 45±5° C. and stirring for 15-20 minutes at 45±5° C. The reaction mass was cooled to 22±2° C. over a period of 3-4 hours and then stirred for 4 to 5 hours at 22±2° C. The resulting mass was then filtered through Buchner flask and the solid was washed with 4 ml of acetone, followed by suction drying the product for 30 minutes using vacuum and then drying the product at 40±5° C. in a vacuum oven under vacuum for 4 hours.

### Example 12

### Preparation of Tapentadol hydrochloride

A mixture of aluminium chloride (11.5 g), thiourea (5 g) and toluene (40 ml) was stirred for 30 minutes at 25±2° C., followed by the addition of (−)-(2R,3R)-[3-(3-methoxyphenyl)-2-methylpentyl]-dimethylamine (5 g) and heating the reaction mass at 110-115° C. The resulting mass was stirred for 2-3 hours at 110-115° C., followed by cooling the mass to 10-15° C. Water (50 ml) and ammonia solution (50 ml) were added to the cooled mass to adjust the pH to 8.0 to 9.0 and the resulting mixture was stirred for 30 minutes. The reaction mass was then filtered through a hyflo bed, and the bed was washed with water (25 ml) and toluene (25 ml). The layers were separated and the aqueous layer was extracted with toluene (25 ml). The organic layers were combined and washed with water (50 ml), followed by distillation of solvents at 50-55° C. under vacuum. The resulting mass was distilled under high vacuum at 50±2° C. for 1 hour to remove toluene substantially completely, isopropyl alcohol (20 ml) was added to the resulting oil and then cooled to 0-5° C. To the resulting mass was added 16% isopropyl alcoholic-HCl (5 ml) followed by stirring for 2 hours at 0-5° C. The reaction mass was filtered through a Buchner flask and the resulting product was dried at 40-45° C. under vacuum for 4 hours to produce 3.35 g of tapentadol hydrochloride [SOR=(−)-30.1° at 20° C., c=1 methanol].

### Example 13

### Preparation of Tapentadol hydrochloride

48% Hydrobromic acid (15 ml) was added to (−)-(2R,3R)-[3-(3-methoxyphenyl)-2-methylpentyl]-dimethylamine (5 g), the mixture was heated at 110-115° C. and then stirred for 6 hours at 110-115° C. The resulting mass was cooled to 10-15° C., followed by the addition of water (10 ml), ethyl acetate (50 ml) and ammonia solution (15 ml) to adjust the pH to 8 to 9. The resulting mixture was stirred for 30 minutes, followed by the separation of the layers and extracting the aqueous layer with ethyl acetate (25 ml). The combined organic layer was washed with water (25 ml), followed by distillation of solvents at 50-55° C. under vacuum and then high vacuum distillation at 50±2° C. for 1 hour to remove ethyl acetate completely. Isopropyl alcohol (20 ml) was added to the resulting oily mass, the resulting mixture was cooled to 0-5° C., followed by the addition of 16% isopropyl alcoholic-HCl (5 ml) and stirring for 2 hours at 0-5° C. The reaction mass was filtered through the Buchner flask and the solid was dried at 40-45° C. under vacuum for 4 hours to produce to produce 4.4 g of tapentadol hydrochloride (Purity by HPLC: 99.69%).

### Example 14

### Preparation of Tapentadol hydrochloride

48% Hydrobromic acid (15 ml) was added to (−)-(2R,3R)-[3-(3-methoxyphenyl)-2-methylpentyl]-dimethylamine (5 g), the mixture was heated at 110-115° C. and then stirred for 6 hours at 110-115° C. The resulting mass was cooled to 10-15° C., followed by the addition of water (10 ml), ethyl acetate (50 ml) and ammonia solution (25 ml) to adjust the pH to 8 to 9. The resulting mixture was stirred for 30 minutes, followed by the separation of the layers and extracting the aqueous layer with ethyl acetate (25 ml). The combined organic layer was washed with water (25 ml), followed by distillation of solvents at 50-55° C. under vacuum and then high vacuum distillation at 50±2° C. for 1 hour to remove ethyl acetate completely. Ethyl acetate (50 ml) was added to the resulting oily mass, followed by the addition of 16% isopropyl alcoholic-HCl (10 ml) and stirring for 20 minutes. The reaction mixture was cooled to 0-5° C. and then stirred for 3 hours. The reaction mass was filtered through the Buchner flask, the solid was washed with ethyl acetate (5 ml) and then dried at 40-45° C. under vacuum for 2 hours to produce 4.83 g of tapentadol hydrochloride (Purity by HPLC: 99.87%).

### Example 15

### Preparation of Tapentadol hydrochloride

48% Hydrobromic acid (15 ml) was added to (−)-(2R,3R)-[3-(3-methoxyphenyl)-2-methylpentyl]-dimethylamine (5 g), the mixture was heated at 110-115° C. and then stirred for 6 hours at 110-115° C. The resulting mass was cooled to 10-15° C., followed by the addition of water (10 ml), ethyl acetate (50 ml) and ammonia solution (25 ml) to adjust the pH to 8 to 9. The resulting mixture was stirred for 30 minutes, followed by the separation of the layers and extracting the aqueous layer with ethyl acetate (25 ml). The combined organic layer was washed with water (25 ml), followed by distillation of solvents at 50-55° C. under vacuum and then high vacuum distillation at 50±2° C. for 1 hour to remove ethyl acetate substantially completely. Isopropyl alcohol (35 ml) was added to the resulting oily mass, followed by the addition of aqueous hydrochloric acid (2.5 ml) and stirring for 20 minutes. The reaction mixture was cooled to 0-5° C. and then stirred for 3 hours. The reaction mass was filtered through the Buchner flask, the solid was washed with isopropyl alcohol (5 ml) and then dried at 40-45° C. under vacuum for 2 hours to produce 3.9 g of tapentadol hydrochloride (Purity by HPLC: 99.97%).

### Example 16

### Preparation of Tapentadol hydrochloride

48% Hydrobromic acid (15 ml) was added to (−)-(2R,3R)-[3-(3-methoxyphenyl)-2-methylpentyl]-dimethylamine (5 g), the mixture was heated at 110-115° C. and then stirred for 6 hours at 110-115° C. The resulting mass was cooled to 10-15° C., followed by the addition of water (10 ml), ethyl acetate (50 ml) and ammonia solution (25 ml) to adjust the pH to 8 to 9. The resulting mixture was stirred for 30 minutes, followed by the separation of the layers and extracting the aqueous layer with ethyl acetate (25 ml). The combined organic layer was washed with water (25 ml), followed by distillation of solvents at 50-55° C. under vacuum and then high vacuum distillation at 50±2° C. for 1 hour to remove ethyl acetate substantially completely. Acetone (40 ml) was added to the resulting oily mass, followed by the addition of 16% isopropyl alcoholic hydrochloric acid (10 ml) and stirring for 20 minutes. The reaction mixture was cooled to 0-5° C. and then stirred for 3 hours. The reaction mass was filtered through the Buchner flask, the solid was washed with acetone (5 ml) and then dried at 40-45° C. under vacuum for 2 hours to produce 4.65 g of tapentadol hydrochloride (Purity by HPLC: 99.97%).

### Example 17

### Preparation of Tapentadol hydrochloride

48% Hydrobromic acid (15 ml) was added to (−)-(2R,3R)-[3-(3-methoxyphenyl)-2-methylpentyl]-dimethylamine (5 g), the mixture was heated at 110-115° C. and then stirred for 6 hours at 110-115° C. The resulting mass was cooled to 10-15° C., followed by the addition of water (10 ml), ethyl acetate (50 ml) and ammonia solution (25 ml) to adjust the pH to 8 to 9. The resulting mixture was stirred for 30 minutes, followed by the separation of the layers and extracting the aqueous layer with ethyl acetate (25 ml). The combined organic layer was washed with water (25 ml), followed by distillation of solvents at 50-55° C. under vacuum and then high vacuum distillation at 50±2° C. for 1 hour to remove ethyl acetate completely. Dichloromethane (40 ml) was added to the resulting oily mass, followed by the addition of 16% isopropyl alcoholic hydrochloric acid (10 ml) and stirring for 20 minutes. The reaction mixture was cooled to 0-5° C. and then stirred for 3 hours. The reaction mass was filtered through the Buchner flask, the solid was washed with dichloromethane (5 ml) and then dried at 40-45° C. under vacuum for 2 hours to produce 4.0 g of tapentadol hydrochloride (Purity by HPLC: 99.97%).

### Example 18

### Preparation of Tapentadol hydrochloride

48% Hydrobromic acid (15 ml) was added to (−)-(2R,3R)-[3-(3-methoxyphenyl)-2-methylpentyl]-dimethylamine (5 g), the mixture was heated at 110-115° C. and then stirred for 6 hours at 110-115° C. The resulting mass was cooled to 10-15° C., followed by the addition of water (10 ml), ethyl acetate (50 ml) and ammonia solution (25 ml) to adjust the pH to 8 to 9. The resulting mixture was stirred for 30 minutes, followed by the separation of the layers and extracting the aqueous layer with ethyl acetate (25 ml). The combined organic layer was washed with water (25 ml), followed by distillation of solvents at 50-55° C. under vacuum and then high vacuum distillation at 50±2° C. for 1 hour to remove ethyl acetate substantially completely. Ethyl acetate (50 ml) was added to the resulting oily mass, followed by the addition of aqueous hydrochloric acid (10 ml) and stirring for 20 minutes. The reaction mixture was cooled to 0-5° C. and then stirred for 3 hours. The reaction mass was filtered through the Buchner flask, the solid was washed with ethyl acetate (5 ml) and then dried at 40-45° C. under vacuum for 2 hours to produce 4.65 g of tapentadol hydrochloride (Purity by HPLC: 99.93%).

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A resolution process for the preparation of (−)-(2R,3R)-1-dimethylamino-3-(3-methoxyphenyl)-2-methylpentan-3-ol of formula III: 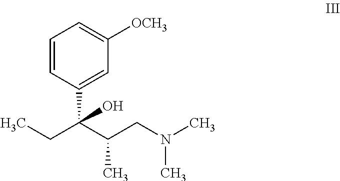 [SMILES: CC[C@](O)(c1cccc(OC)c1)[C@@H](C)CN(C)C]
or an acid addition salt thereof, comprising:
a) treating an enantiomeric pair (2R,3R)/(2S,3S)-1-dimethylamino-3-(3-methoxyphenyl)-2-methylpentan-3-ol or an acid addition salt thereof with (αS)-6-methoxy-α-methyl-2-naphthaleneacetic acid (S-naproxen) in a first solvent to produce a reaction mass containing the diastereomeric mixture of desired diastereomeric salt, (2R,3R)-1-dimethylamino-3-(3-methoxyphenyl)-2-methylpentan-3-ol S-naproxen salt, and undesired diastereomeric salt, (2S,3S)-1-dimethylamino-3-(3-methoxyphenyl)-2-methylpentan-3-ol S-naproxen salt;
b) separating the undesired diastereomeric salt from the diastereomeric mixture obtained in step-(a) to produce the desired diastereomeric salt; and
c) neutralizing the desired diastereomeric salt obtained in step-(b) with a base in a second solvent to produce enantiomerically pure (−)-(2R,3R)-1-dimethylamino-3-(3-methoxyphenyl)-2-methylpentan-3-ol of formula III, and optionally converting the enantiomerically pure compound of formula III obtained into an acid addition salt thereof.

2. The process of claim 1, wherein the first solvent used in step-(a) is selected from the group consisting of water, an alcohol, a ketone, a cyclic ether, an aliphatic ether, a hydrocarbon, a chlorinated hydrocarbon, a nitrile, an ester, a polar aprotic solvent, and mixtures thereof; wherein the second solvent used in step-(c) is selected from the group consisting of water, an alcohol, a ketone, a cyclic ether, an aliphatic ether, a hydrocarbon, a chlorinated hydrocarbon, a nitrile, an ester, and mixtures thereof; and wherein the base used in step-(c) is selected from the group consisting of triethylamine, trimethylamine, dimethyl amine, tert-butyl amine, aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide.

3. The process of claim 2, wherein the first solvent is selected from the group consisting of water, methanol, ethanol, isopropanol, acetonitrile, and mixtures thereof; wherein the second solvent is selected from the group consisting of water, methylene chloride, n-hexane, n-heptane, cyclohexane, toluene, xylene, and mixtures thereof; and wherein the base used in step-(c) is selected from the group consisting of aqueous ammonia, sodium hydroxide, potassium hydroxide, sodium carbonate and potassium carbonate.

4. The process of claim 1, wherein the reaction in step-(a) is carried out at a temperature of −20° C. to the reflux temperature of the solvent used; wherein the separation of diastereomers in step-(b) is carried out by fractional crystallization; wherein the pH of the reaction mass in step-(c) is adjusted to above 7; and wherein the enantiomerically pure (−)-(2R,3R)-1-dimethylamino-3-(3-methoxyphenyl)-2-methylpentan-3-ol of formula III formed in step-(c) is isolated from a suitable organic solvent by cooling, seeding, partial removal of the solvent from the solution, by adding an anti-solvent to the solution, evaporation, vacuum distillation, or a combination thereof.

5. The process of claim 4, wherein the reaction in step-(a) is carried out at a temperature of about 0° C. to about 60° C.; and wherein the pH of the reaction mass in step-(c) is adjusted between 9 and 10.

6. A resolution process for the preparation of (−)-(2R,3R)-[3-(3-methoxyphenyl)-2-methylpentyl]-dimethylamine of formula II: 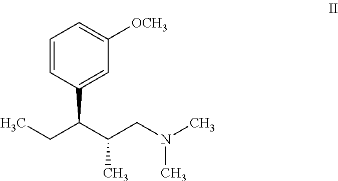 [SMILES: CC[C@@H](c1cccc(OC)c1)[C@@H](C)CN(C)C]
or an acid addition salt thereof, comprising:
a) treating an enantiomeric pair (2R,3R)/(2S,3S)-[3-(3-methoxyphenyl)-2-methylpentyl]-dimethylamine or an acid addition salt thereof with an optically active acid in a first solvent to produce a reaction mass containing the diastereomeric mixture;
b) separating the desired diastereomeric salt from the diastereomeric mixture obtained in step-(a); and
c) neutralizing the desired diastereomeric salt obtained in step-(b) with a base in a second solvent to produce enantiomerically pure (−)-(2R,3R)-[3-(3-methoxyphenyl)-2-methylpentyl]-dimethylamine of formula II, and optionally converting the enantiomerically pure compound of formula II obtained into an acid addition salt thereof.

7. The process of claim 6, wherein the optically active acid used in step-(a) is selected from the group consisting of S-naproxen, (−)-di-p-toluoyl-L-tartaric acid, (+)-di-p-toluoyl-D-tartaric acid, (−)-dibenzoyl-L-tartaric acid, (+)-dibenzoyl-D-tartaric acid, and hydrates thereof; wherein the first solvent used in step-(a) is selected from the group consisting of water, an alcohol, a ketone, a cyclic ether, an aliphatic ether, a hydrocarbon, a chlorinated hydrocarbon, a nitrile, an ester, a polar aprotic solvent, and mixtures thereof; wherein the second solvent used in step-(c) is selected from the group consisting of water, an alcohol, a ketone, a cyclic ether, an aliphatic ether, a hydrocarbon, a chlorinated hydrocarbon, a nitrile, an ester, and mixtures thereof; and wherein the base used in step-(c) is selected from the group consisting of triethylamine, trimethylamine, dimethyl amine, tert-butyl amine, aqueous ammonia, sodium hydroxide, calcium hydroxide, magnesium hydroxide, potassium hydroxide, lithium hydroxide, sodium carbonate, potassium carbonate, sodium bicarbonate, potassium bicarbonate, lithium carbonate, sodium tert-butoxide, sodium isopropoxide and potassium tert-butoxide.

8. The process of claim 7, wherein the optically active acid used in step-(a) is selected from the group consisting of (−)-di-p-toluoyl-L-tartaric acid and S-naproxen; wherein the first solvent used in step-(a) is selected from the group consisting of water, methanol, ethanol, isopropanol, acetonitrile, and mixtures thereof; wherein the second solvent used in step-(c) is selected from the group consisting of water, methylene chloride, n-hexane, n-heptane, cyclohexane, toluene, xylene, and mixtures thereof; and wherein the base used in step-(c) is selected from the group consisting of aqueous ammonia, sodium hydroxide, potassium hydroxide, sodium carbonate and potassium carbonate.

9. The process of claim 6, wherein the reaction in step-(a) is carried out at a temperature of −20° C. to the reflux temperature of the solvent used; wherein the separation of diastereomers in step-(b) is carried out by fractional crystallization; wherein the pH of the reaction mass in step-(c) is adjusted to above 7; and wherein the enantiomerically pure (−)-(2R,3R)-[3-(3-methoxyphenyl)-2-methylpentyl]-dimethylamine of formula II formed in step-(c) is isolated from a suitable organic solvent by cooling, seeding, partial removal of the solvent from the solution, by adding an anti-solvent to the solution, evaporation, vacuum distillation, or a combination thereof.

10. The process of claim 9, wherein the reaction in step-(a) is carried out at a temperature of about 0° C. to about 80° C.; and wherein the pH of the reaction mass in step-(c) is adjusted between 7 and 8.

11. A process for preparing tapentadol, 3-[(1R,2R)-3-(dimethylamino)-1-ethyl-2-methylpropyl]phenol, of formula I: 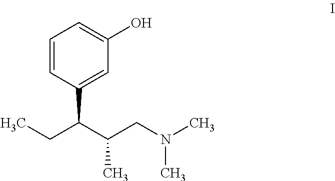 [SMILES: CC[C@@H](c1cccc(O)c1)[C@@H](C)CN(C)C]
or a pharmaceutically acceptable salt thereof, comprising
a) reacting an enantiomeric pair (2R,3R)/(2S,3S)-1-dimethylamino-3-(3-methoxyphenyl)-2-methylpentan-3-ol or an acid addition salt thereof with trifluoroacetic anhydride in a first solvent to produce a reaction mass;
b) hydrogenating the reaction mass obtained in step-(a) in the presence of a hydrogenation catalyst to produce an enantiomeric pair (2R,3R)/(2S,3S)-[3-(3-methoxyphenyl)-2-methylpentyl]-dimethylamine or an acid addition salt thereof;
c) resolving the enantiomeric pair obtained in step-(b) with a suitable optically active acid to produce an enantiomerically pure (−)-(2R,3R)-[3-(3-methoxyphenyl)-2-methylpentyl]-dimethylamine of formula II or an acid addition salt thereof, wherein the optically active acid is selected from the group consisting of optically active di-aroyl-tartaric acid, S-naproxen, malic acid, mandelic acid and its derivatives, and camphorsulphonic acid and its derivatives;
d) demethylating the enantiomerically pure compound of formula II obtained in step-(c) using a demethylating agent in a second solvent to produce tapentadol of formula I, and optionally converting the tapentadol of formula I obtained into a pharmaceutically acceptable salt thereof; and
e) optionally, purifying the tapentadol or a pharmaceutically acceptable salt thereof obtained in step-(d) using a third solvent to produce highly pure tapentadol or a pharmaceutically acceptable salt thereof.

12. The process of claim 11, wherein the first solvent used in step-(a) is selected from the group consisting of water, an alcohol, a ketone, a cyclic ether, an aliphatic ether, a hydrocarbon, a chlorinated hydrocarbon, a nitrile solvent, and mixtures thereof; wherein the second solvent used in step-(d) is selected from the group consisting of water, an alcohol, a ketone, a cyclic ether, an aliphatic ether, a hydrocarbon, a chlorinated hydrocarbon, a nitrile solvent, and mixtures thereof; and wherein the third solvent used in step-(e) is selected from the group consisting of water, an alcohol, a ketone, and mixtures thereof.

13. The process of claim 12, wherein the first solvent used in step-(a) is selected from the group consisting of methanol, ethanol, isopropanol, acetonitrile, tetrahydrofuran, 2-methyl tetrahydrofuran, and mixtures thereof; wherein the second solvent used in step-(d) is toluene; and wherein the third solvent used in step-(e) is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, and mixtures thereof.

14. The process of claim 11, wherein the hydrogenation catalyst used in step-(b) is selected from the group consisting of palladium hydroxide, palladium on carbon, platinum on carbon, platinum oxide, rhodium on carbon, rhodium on alumina; wherein the demethylating agent used in step-(d) is selected from the group consisting of hydrobromic acid, aluminum chloride/thiourea, aluminium triiodide/tetrabutylammonium iodide and ClBH₂.Me₂S; and wherein the hydrobromic acid in step-(d) is used in the molar ratio of about 2 to 10 volumes per 1 gm of the (−)-(2R,3R)-[3-(3-methoxyphenyl)-2-methylpentyl]-dimethylamine of formula II.

15. The process of claim 14, wherein the hydrogenation catalyst used in step-(b) is palladium on carbon; wherein the demethylating agent used in step-(d) is hydrobromic acid; and wherein the hydrobromic acid in step-(d) is used in the molar ratio of about 3 to 4 volumes per 1 gm of the (−)-(2R,3R)-[3-(3-methoxyphenyl)-2-methylpentyl]-dimethylamine of formula II.

16. The process of claim 11, wherein the reaction in step-(a) is carried out at a temperature of −20° C. to 50° C.; wherein the hydrogenation reaction in step-(b) is carried out at a temperature of 0° C. to the reflux temperature of the solvent used; wherein the hydrogenation reaction in step-(b) is carried out under hydrogen pressure or in the presence of hydrogen transfer reagent; wherein the hydrogenation catalyst in step-(b) is used in the ratio of about 0.05% (w/w) to 10% (w/w) with respect to the enantiomeric pair (2R,3R)/(2S,3S)-1-dimethylamino-3-(3-methoxyphenyl)-2-methylpentan-3-ol; and wherein the reaction in step-(d) is carried out at a temperature of 0° C. to the reflux temperature of the solvent used.

17. The process of claim 16, wherein the reaction in step-(a) is carried out at a temperature of about 0° C. to about 40° C.; wherein the hydrogenation reaction in step-(b) is carried out at a temperature of about 0° C. to about 50° C.; wherein the hydrogen transfer reagent used in step-(b) is selected from the group consisting of formic acid, ammonium formate, sodium formate, trimethylammonium formate and tributylammonium formate; wherein the hydrogenation catalyst is used in the ratio of about 0.5% (w/w) to 2.5% (w/w) with respect to the enantiomeric pair (2R,3R)/(2S,3S)-1-dimethylamino-3-(3-methoxyphenyl)-2-methylpentan-3-ol; and wherein the reaction in step-(d) is carried out at a temperature of about 50° C. to about 120° C.

18. The process of claim 11, wherein the purification in step-(e) is carried out by a process comprising providing a solution of tapentadol or a pharmaceutically acceptable salt thereof in the third solvent, optionally, subjecting the solution to carbon treatment or silica gel treatment; and isolating the highly pure of tapentadol or a pharmaceutically acceptable salt thereof from the solution.

19-20. (canceled)

21. The method of claim 6, wherein the optically active acid in step (a) is (−)-di-p-toluoyl-L-tartaric acid or (−)-Dibenzoyl-L-tartaric acid.

22. The method of claim 6, wherein the optically active acid in step (c) is (−)-di-p-toluoyl-L-tartaric acid or (−)-Dibenzoyl-L-tartaric acid.
